(19) United States
(12) Patent Application Publication
(10) Pub. No.: US 2026/0134970 A1
(43) Pub. Date: May 14, 2026

(54) AI ASSISTED ORTHOPEDIC SURGERY SYSTEM AND METHOD

(71) Applicant: AEVUMED, INC., Malvern, PA (US)

(72) Inventors: Miles O. Curtis, Philadelphia, PA (US); Robert P. Douglas, Bryn Mawr, PA (US)

(21) Appl. No.: 19/388,469

(22) Filed: Nov. 13, 2025

Related U.S. Application Data
(60) Provisional application No. 63/720,005, filed on Nov. 13, 2024.

(51) Int. Cl. G16H 20/40; A61B 34/10; A61B 90/00; G06T 7/00; G06T 11/60; G06V 10/764; G06V 10/774; G06V 10/82; G06V 20/50

(52) U.S. Cl. CPC G16H 20/40; A61B 34/10; G06T 7/0016; G06T 11/60; G06V 10/764; G06V 10/774; G06V 10/82; G06V 20/50; A61B 2034/104; A61B 2034/105; A61B 2034/107; A61B 2090/064; G06T 2200/24; G06T 2207/20081; G06T 2207/20084; G06T 2207/30004; G06T 2210/41; G06V 2201/03

(57) ABSTRACT

An AI assisted orthopedic surgery system includes a computing device with a neural network trained on anatomical and medical data that generates AR overlays and outputs treatment options including tissue anchor and suture recommendations on live video streams from arthroscopic devices during surgical procedures. Also an AI Assisted orthopedic surgery computer readable media or software is disclosed. Further disclosed is a pressure sensing tissue anchor.

### CROSS-REFERENCE TO RELATED APPLICATIONS

This application claims priority to U.S. Provisional Application No. 63/720,005, filed on Nov. 13, 2024, incorporated herein by reference in its entirety.

### BACKGROUND OF THE INVENTION

Orthopedic surgeries often involve complex and precise procedures to repair or restore musculoskeletal function. In arthroscopic surgeries, especially in the shoulder, knee, and hip, it is crucial for surgeons to accurately identify, position, and secure tissue anchors and sutures to effectively treat tendon tears or other injuries.

Traditional methods of performing these surgeries rely heavily on the surgeon's expertise and ability to interpret real-time arthroscopic video, often without any augmented guidance. This can pose challenges in determining the optimal placement of anchors, the type of anchor to use, the type and pattern of sutures, and the correct tension to be applied to sutures, especially when visual access is limited and dependent on live camera feeds.

Advances in artificial intelligence (AI) and augmented reality (AR) have shown potential in assisting complex medical procedures by enhancing visualization and decision-making. However, current solutions in orthopedic surgery primarily focus on preoperative planning or diagnostic assistance and are not integrated into a real-time, intraoperative setting to provide active guidance during surgery.

Thus, there is a need in the art for AI assisted orthopedic surgery systems and methods in order to enhance treatments for tendon tears and other injuries. The present invention satisfies that need.

### SUMMARY OF THE INVENTION

In one aspect, a method for assisting orthopedic surgery includes the steps of collecting imaging data of a region of interest of a subject, analyzing the imaging data with a neural network, wherein the neural network is trained to identify anatomical structures, surgical devices and injuries, classifying, by the neural network, at least one of anatomical structures, surgical devices, and injuries in the imaging data, generating an overlay for the video with one or more graphical representations of any classified at least one anatomical structures, surgical devices and injuries, and outputting one or more treatment options for any identified injury.

In some embodiments, the one or more treatment options are selected from the group consisting of non-implant repair, surgical device requirement, surgical device type, closure device requirement, closure device type, tissue anchor requirement, tissue anchor type, tissue anchor number, one or more tissue anchor locations, tissue anchor layout, suture type, suture locations, and suture configuration.

In some embodiments, the overlay displays one or more treatment options comprising graphical representations of one or more non-implant repairs, one or more surgical devices, closure devices, tissue anchors or suture applied to treat an identified injury. In some embodiments, the overlay displays one or more graphical representations of suture attached to one or more tissue anchors applied to treat an identified injury. In some embodiments, graphically represented anchor or suture is numbered, or labelled with a position relative to the region of interest, injury, or anatomical structure.

In some embodiments, the method further includes the steps of receiving one or more inputs from a user, and modifying at least one of the overlay or the one or more treatment options based on the inputs from the user. In some embodiments, the one or more inputs are selected from the group consisting of changes to the overlay, addition or removal of a closure device, addition or removal of an anchor, addition or removal of suture, change in closure device location, change in anchor location, change in suture location or configuration, addition or removal of an identified anatomical structure, refining the location or representation of an identified anatomical structure, addition or removal of an identified injury, refining the location or representation of an identified injury preoperative MRI data, patient examination data, quality of tendon, excursion, characteristics of tear, degree of retraction, and superior migration.

In some embodiments, the neural network is trained with medical training data selected from the group consisting of medical imaging data, raw arthroscopic video recordings, edited arthroscopic video recordings with manually added overlays, edited arthroscopic video recordings with identified anatomical structures or injuries, MRI data, preoperative MRI data, postoperative MRI data, possible treatment options, treatment options employed in a repair, closure devices employed in a repair, anchors employed in a repair, anchor layout employed in a repair, suture used in a repair, suture configuration used in a repair, data scraped from online medical journal publications, case registries, patient information, and treatment outcomes.

In some embodiments, the method further includes the steps of collecting patient information of the subject, analyzing the patient information with the neural network, and modifying at least one of the overlay or the one or more treatment options based on the results from the neural network. In some embodiments, the method further includes the steps of predicting post-operative outcomes based on the one or more treatment options and the patient information, and displaying the predicted outcomes in the overlay to provide real-time guidance to the user.

In some embodiments, the method further includes the steps of calculating an anatomical structure identification confidence score, and displaying the confidence score in the overlay. In some embodiments, the method further includes the steps of calculating a tear percentage of an identified injury, and displaying the tear percentage in the overlay. In some embodiments, the method further includes the steps of monitoring real-time changes in the region of interest with the neural network, and modifying at least one of the overlay or the one or more treatment options based on the changes. In some embodiments, the real-time changes comprise any of tissue deformation, tissue color change, movement of anatomical structures, movement of surgical devices, movement of closure devices, anchor position, suture tension, and suture position. In some embodiments, the imaging data comprises at least one of camera image, sensor image, camera video, sensor video, live camera video, live sensor video, endoscopic image, endoscopic video, live endoscopic video, prerecorded endoscopic video, arthroscopic image, arthroscopic video, live arthroscopic video, prerecorded arthroscopic video, non-arthroscopic video, wearable device camera image, wearable device sensor image, wearable device camera video, wearable device sensor video, smart glasses camera image, smart glasses sensor image, smart glasses camera video, smart glasses sensor video, live smart glasses camera video, live smart glasses sensor video, surgical camera, operating room camera.

In some embodiments, the method further includes the step of receiving signals from one or more pressure sensing tissue anchors configured to measure tension of suture attached to the anchor. In some embodiments, the method further includes the step of displaying the measured tension in the overlay. In some embodiments, the method further includes the step of modifying the one or more treatment options based on the measured tension. In some embodiments, the method further includes the step of measuring change in suture tension over time. In some embodiments, a change in suture tension over time may be representative of suture tearing through tendon, suture cutting through bone, and tendon healing.

In some embodiments, the method further includes the step of outputting one or more alternative treatment options based on an input indicative of an issue with the one or more treatment options.

In one aspect, an AI assisted orthopedic surgery system includes a non-transitory computer-readable medium with instructions stored thereon, that when executed by a processor perform the steps of collecting imaging data of a region of interest of a subject, analyzing the imaging data with a neural network, wherein the neural network is trained to identify anatomical structures, surgical devices and injuries, classifying, by the neural network, at least one of anatomical structures, surgical devices, and injuries in the imaging data, generating an overlay for the video with one or more graphical representations of any classified at least one anatomical structures, surgical devices and injuries, and outputting one or more treatment options for any identified injury.

In some embodiments, the system is configured to connect between an arthroscopic camera and a display. In some embodiments, the system includes one or more pressure sensing tissue anchors connected to the system configured to measure tension of suture attached to the anchor.

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements, and in which:

FIG. 1 is a diagram of a computing device according to one embodiment.

FIG. 2 is a diagram depicting an exemplary AI Assisted orthopedic surgery system (AI-OSS) and training model according to one embodiment.

FIG. 3 is an image depicting an exemplary overlay or user interface (UI) for an AI-OSS according to one embodiment.

FIG. 4 depicts an exemplary pressure sensing tissue anchor according to one embodiment.

### DETAILED DESCRIPTION

It is to be understood that the figures and descriptions of the present invention have been simplified to illustrate elements that are relevant for a clear understanding of the present invention, while eliminating, for the purpose of clarity, many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

In some aspects of the present invention, software executing the instructions provided herein may be stored on a non-transitory computer-readable medium, wherein the software performs some or all of the steps of the present invention when executed on a processor.

Aspects of the invention relate to algorithms executed in computer software. Though certain embodiments may be described as written in particular programming languages, or executed on particular operating systems or computing platforms, it is understood that the system and method of the present invention is not limited to any particular computing language, platform, or combination thereof. Software executing the algorithms described herein may be written in any programming language known in the art, compiled or interpreted, including but not limited to C, C++, C#, Objective-C, Java, JavaScript, MATLAB, Python, PHP, Perl, Ruby, R or Visual Basic. It is further understood that elements of the present invention may be executed on any acceptable computing platform, including but not limited to a server, a cloud instance, a workstation, a thin client, a mobile device, an embedded microcontroller, a television, or any other suitable computing device known in the art.

Parts of this invention are described as software running on a computing device. Though software described herein may be disclosed as operating on one particular computing device (e.g. a dedicated server or a workstation), it is understood in the art that software is intrinsically portable and that most software running on a dedicated server may also be run, for the purposes of the present invention, on any of a wide range of devices including desktop or mobile devices, laptops, tablets, smartphones, watches, wearable electronics or other wireless digital/cellular phones, televisions, cloud instances, embedded microcontrollers, thin client devices, or any other suitable computing device known in the art.

Similarly, parts of this invention are described as communicating over a variety of wireless or wired computer networks. For the purposes of this invention, the words “network”, “networked”, and “networking” are understood to encompass wired Ethernet, fiber optic connections, wireless connections including any of the various 802.11 standards, cellular WAN infrastructures such as 3G, 4G/LTE, or 5G networks, Bluetooth®, Bluetooth® Low Energy (BLE) or Zigbee® communication links, or any other method by which one electronic device is capable of communicating with another. In some embodiments, elements of the networked portion of the invention may be implemented over a Virtual Private Network (VPN).

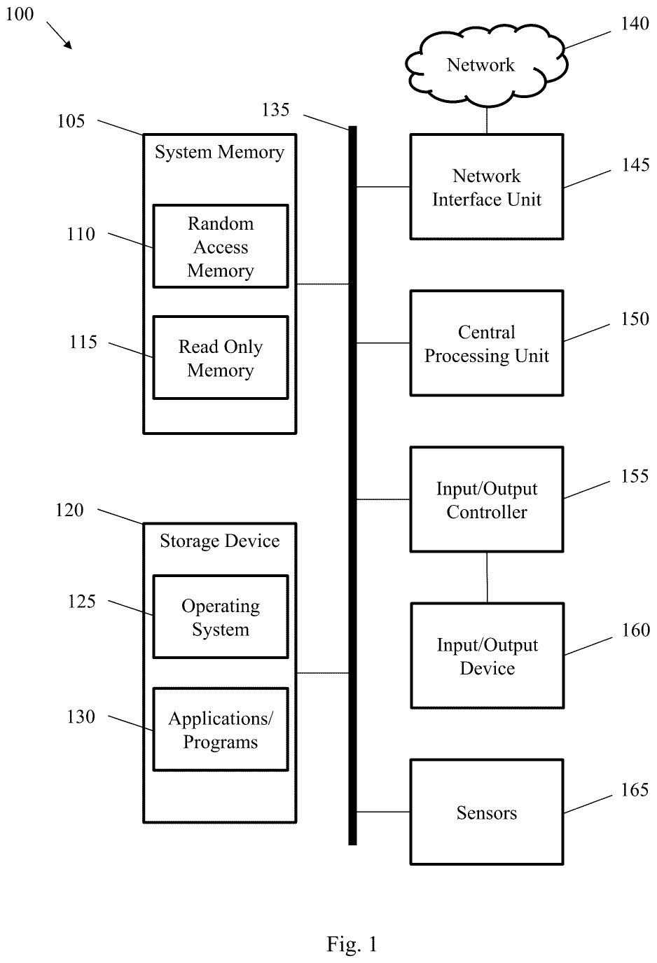

FIG. 1 and the following discussion are intended to provide a brief, general description of a suitable computing environment in which the invention may be implemented according to one embodiment. While the invention is described above in the general context of program modules that execute in conjunction with an application program that runs on an operating system on a computer, those skilled in the art will recognize that the invention may also be implemented in combination with other program modules.

Generally, program modules include routines, programs, components, data structures, and other types of structures that perform particular tasks or implement particular abstract data types. Moreover, those skilled in the art will appreciate that the invention may be practiced with other computer system configurations, including hand-held devices, multiprocessor systems, microprocessor-based or programmable consumer electronics, minicomputers, mainframe computers, and the like. The invention may also be practiced in distributed computing environments where tasks are performed by remote processing devices that are linked through a communications network. In a distributed computing environment, program modules may be located in both local and remote memory storage devices.

FIG. 1 depicts an illustrative computer architecture for a computer 100 for practicing the various embodiments of the invention. The computer architecture shown in FIG. 1 illustrates a conventional personal computer, including a central processing unit 150 (“CPU”), a system memory 105, including a random access memory 110 (“RAM”) and a read-only memory (“ROM”) 115, and a system bus 135 that couples the system memory 105 to the CPU 150. A basic input/output system containing the basic routines that help to transfer information between elements within the computer, such as during startup, is stored in the ROM 115. The computer 100 further includes a storage device 120 for storing an operating system 125, application/program 130, and data. The computer architecture may in certain embodiments physically reside in medical equipment found in hospitals and surgical suites, or otherwise communicatively coupled with the medical equipment.

The storage device 120 is connected to the CPU 150 through a storage controller (not shown) connected to the bus 135. The storage device 120 and its associated computer-readable media provide non-volatile storage for the computer 100. Although the description of computer-readable media contained herein refers to a storage device, such as a hard disk or CD-ROM drive, it should be appreciated by those skilled in the art that computer-readable media can be any available media that can be accessed by the computer 100.

By way of example, and not to be limiting, computer-readable media may comprise computer storage media. Computer storage media includes volatile and non-volatile, removable and non-removable media implemented in any method or technology for storage of information such as computer-readable instructions, data structures, program modules or other data. Computer storage media includes, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by the computer.

According to various embodiments of the invention, the computer 100 may operate in a networked environment using logical connections to remote computers through a network 140, such as TCP/IP network such as the Internet or an intranet. The computer 100 may connect to the network 140 through a network interface unit 145 connected to the bus 135. It should be appreciated that the network interface unit 145 may also be utilized to connect to other types of networks and remote computer systems.

The computer 100 may also include an input/output controller 155 for receiving and processing input from a number of input/output devices 160, including medical equipment found in hospitals and surgical suites such as cameras and imaging equipment, or more generally a keyboard, a mouse, a display, a touchscreen, a handheld phone, a camera, a microphone, a controller, a joystick, or other type of input device. Similarly, the input/output controller 155 may provide output to a display screen, a printer, a speaker, or other type of output device such as a wearable device (e.g., smart glasses) or a heads-up display (HUD). The computer 100 can connect to the input/output device 160 via a wired connection including, but not limited to, fiber optic, Ethernet, or copper wire or wireless means including, but not limited to, Wi-Fi, Bluetooth, Near-Field Communication (NFC), infrared, or other suitable wired or wireless connections.

As mentioned briefly above, a number of program modules and data files may be stored in the storage device 120 and/or RAM 110 of the computer 100, including an operating system 125 suitable for controlling the operation of a networked computer. The storage device 120 and RAM 110 may also store one or more applications/programs 130. In particular, the storage device 120 and RAM 110 may store an application/program 130 for providing a variety of functionalities to a user. For instance, the application/program 130 may comprise many types of programs such as a word processing application, a spreadsheet application, a desktop publishing application, a database application, a gaming application, internet browsing application, electronic mail application, messaging application, and the like. According to an embodiment of the present invention, the application/program 130 comprises a multiple functionality software application for providing word processing functionality, slide presentation functionality, spreadsheet functionality, database functionality and the like.

The computer 100 in some embodiments can include a variety of sensors 165 for monitoring the environment surrounding and the environment internal to the computer 100. These sensors 165 can include sensors deployed in medical equipment found in hospitals and surgical suites such as cameras and imaging equipment, or other sensors such as a Global Positioning System (GPS) sensor, a photosensitive sensor, a gyroscope, a magnetometer, thermometer, a proximity sensor, an accelerometer, a microphone, biometric sensor, barometer, humidity sensor, radiation sensor, or any other suitable sensor.

In some embodiments, computer 100 comprises the components necessary to enable the AI-assisted orthopedic surgery system and method disclosed herein. These components comprise, but are not limited to, one or more neural networks with one or more models trained on medical training data including anatomical, surgical and patient data. The processes of the system and method are optimized through multiple layers, including an input layer for receiving real-time imaging data from arthroscopic camera, hidden layers for analyzing anatomical structures, surgical devices, and injuries, and an output layer that generates live overlays, identifiers and procedural recommendations. Additionally, computer 100 includes a transformer-based natural language processing (NLP) model, configured to receive and interpret surgeon inputs and queries, and adjust procedural recommendations accordingly or chat with the surgeon (e.g., a chatbot). In some embodiments, the chatbot or chat is displayed in the UI and provides guidance and/or feedback to a surgeon during a procedure. In some embodiments, the chatbot provides text feedback that is separate from (sent to another display or handheld) or alongside the UI (e.g., displayed in the arthroscopic overlay). In some embodiments, the text or chat of the chatbot is vocalized to the surgeon, and can receive voice commands. In some embodiments, the chatbot is a standalone app using the systems and methods disclosed herein to provide surgical guidance or critique on medical imaging and case data. Users may chat with the chatbot in the app or communicate with the video AI via a transformer. In some embodiments, the model is trained on patient-specific data, including demographic information and medical history, to enable personalized, data-driven guidance during surgery. The computer 100 further facilitates communication between the neural network and the NLP model, allowing real-time generation of augmented reality overlays based on the surgeon inputs to guide surgical procedures.

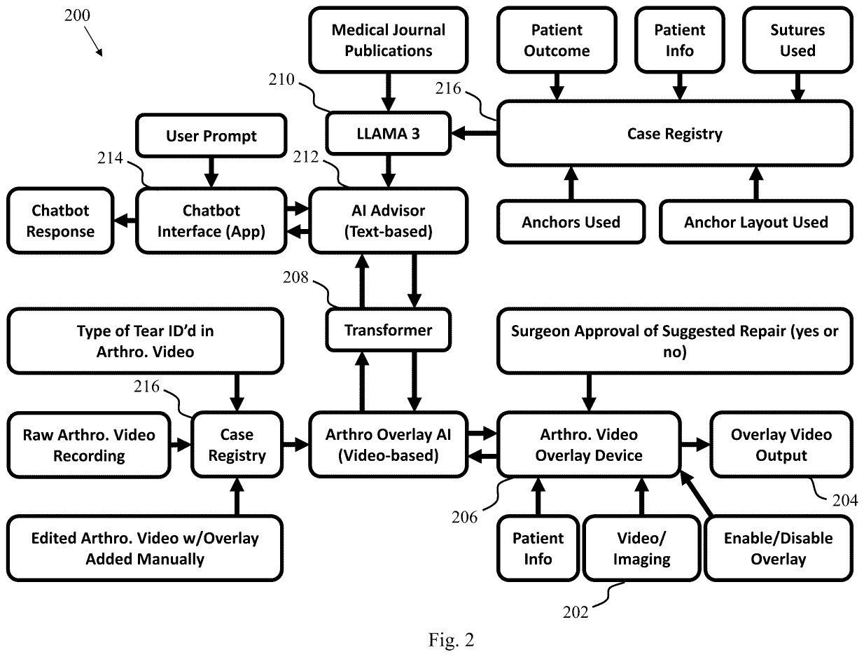

Referring now to FIG. 2, shown is a diagram depicting an exemplary AI Assisted orthopedic surgery system 200 (AI-OSS) and training model. In some embodiments, the disclosed system 200 and method integrates AI and AR technology to enhance orthopedic surgery with treatment recommendations for real-time identified injuries. In some embodiments, AI-OSS leverages imaging or video 202 (e.g., live or pre-recorded arthroscopic video and/or imaging) from arthroscopic equipment to identify anatomical structures and detect injuries, and provides surgeons with visual overlays and/or AR 204 showing potential treatment options including tissue anchor placements, suture types, and configurations. The visual overlays and/or AR 204 may comprise 2D overlays, as well as 3D visualizations or projections of any disclosed features. In some embodiments, the system 200 operates as any of: a standalone device 206, a software package and/or computer readable media on a computing device (e.g., computer 100) providing an AR-enhanced video feed to help guide a surgeon through a procedure. In some embodiments, the system provides AR-enhanced video feed for existing systems (e.g., surgical systems). In some embodiments, the system 200 comprises an AI advisor 212 comprising a chatbot interface 214 as input and output means for the system and providing conversational abilities. In some embodiments, a case registry 214 is produced for a subject aggregating imaging, video, patient information, injuries, and patient outcomes, as well as treatments such as employed sutures, anchors and anchor layouts. By utilizing AI models, including neural networks 208, NLPs, and large language models (LLMs) 210, the disclosed system can query imaging (e.g., live imaging, arthroscopic video, prerecorded video), interpret input data, and provide contextual recommendations, thus supporting surgeons in achieving more precise and effective outcomes.

The disclosed system may receive imaging data in various forms from various sources, including, but not limited to: camera image, sensor image, camera video, sensor video, live camera video, live sensor video, endoscopic image, endoscopic video, live endoscopic video, prerecorded endoscopic video, arthroscopic image, arthroscopic video, live arthroscopic video, prerecorded arthroscopic video, non-arthroscopic video, wearable device camera image, wearable device sensor image, wearable device camera video, wearable device sensor video, smart glasses camera image, smart glasses sensor image, smart glasses camera video, smart glasses sensor video, live smart glasses camera video, live smart glasses sensor video, surgical camera, operating room camera.

The disclosed system is versatile, functioning either as software compatible with many devices or systems, or as a standalone intermediate or middleman device that connects directly to or between imaging equipment and displays. In some embodiments, the system connects to or is interoperable with any medical imaging equipment, software or systems that would be known by one of ordinary level of skill in the art. In some embodiments, the system overlays treatment recommendations, live patient metrics, or patient information over video captured by standard medical imaging equipment. In some embodiments, the system provides an overlay or AR enhanced feed for a pre-recorded arthroscopic video. The disclosed system supports a wide range of orthopedic surgical tasks but also has the potential to learn from each procedure, continuously improving its recommendations based on feedback and case data, and contributing to the advancement of orthopedic surgical practices.

In some embodiments, the AI models are trained to detect or identify surgical devices, anatomical structures or tissue injuries. In some embodiments, the system can detect or identify surgical devices in the region of interest such as closure devices, anchors, staples, suture, grafts, scaffolds, buttons, plates, implants, prosthesis, orthopedic devices, or the like. In some embodiments, the system identifies anatomical structures, such as the rotator cuff. In some embodiments, the system can identify injuries, such as tendon tear, and provide tendon tear information. In some embodiments, the system can identify or recommend one or more treatment options. In some embodiments, the treatment options comprise any of non-implant repair, surgical device requirement, surgical device type, closure device requirement, closure device type, tissue anchor requirement, tissue anchor type, tissue anchor number, one or more tissue anchor locations, tissue anchor layout, suture type (e.g., #2, tapes, etc), suture locations, and suture configuration, pattern, or layout, or recommended suture tension, graft requirement, graft placement, graft number, graft type, graft configuration, scaffold requirement, scaffold placement, scaffold number, scaffold type, scaffold material, scaffold configuration, button requirement, button placement, button number, button type, button configuration, plate requirement, plate placement, plate number, plate type, and plate configuration. Closure devices may include for example skin closure device for closing surgical incisions, zip devices, stitches, staples, adhesives and other devices known in the art for closing surgical incisions. In some embodiments, the system and method may recommend a repair with one or more anchors, devices, sutures, grafts, scaffolds, buttons, plates, etc. However, the system and method may also recommend to not use any device or suture (e.g., a non-implant repair), or no repair at all. The treatment options and/or recommendations may further comprise one or more grafts, including graft type, size and pattern, however recommendations for no-graft treatments may also be provided. In some embodiments, non-implants repairs may include, but are not limited to, clean up tendon, surfaces, or perform an acromioplasty to address impingement, or the like. In some embodiments, the system may receive an input or prompt (e.g., voice command) requesting the treatment options are narrowed based on any of the inputs disclosed herein, including but not limited to, patient data (e.g., demographics, imaging), treatment type, and severity of injury. Further details of training and capabilities of the model are provided in the algorithm training discussed herein.

In some embodiments, the system accepts or receives one or more inputs from a user (e.g., a surgeon), or from another system or software (e.g., an arthroscopic camera, an electronic medical record software, a medical device). In some embodiments, inputs to the system comprise any of patient information, age, gender, height, weight, BMI, patient-specific risk factors, smoker status, medical imaging, MRI scans, preoperative MRI data, Xrays, history of applicable pre-existing conditions, history of previous repairs, live arthroscopic video feed, surgeon approval of suggested repair, manual enable/disable of overlay, pre-operative imaging, surgical device cost data, operative time, intraoperative vital signs, medical imaging data, raw arthroscopic video recordings, edited arthroscopic video recordings with manually added overlays, edited arthroscopic video recordings with identified anatomical structures or injuries, possible treatment options, treatment options employed in a repair, closure devices employed in a repair, anchors employed in a repair, anchor layout employed in a repair, suture used in a repair, suture configuration used in a repair, data scraped from online medical journal publications, case registries, patient information, treatment outcomes, patient examination data, quality of tendon, excursion, characteristics of tear, degree of retraction, and superior migration. In some embodiments, the system receives inputs (e.g., suture tension) from an active implant such as a strain or pressure sensing device (e.g., a pressure sensing tissue anchor disclosed herein). In some embodiments, the system receives one or more inputs from one or more active implants (e.g., sensing tissue anchors), wherein the inputs comprise any of compressive load of tendon on top of implant, temperature, movement, orientation, vibration, bony ingrown into the anchor, pressure on anchor, or the like. In some embodiments, one or more active implants sends feedback or measurements to the disclosed one or more neural networks. Thus, the system may in one embodiment review historical patient imaging data with real time imaging data, and based on the AI analysis modify the treatment plan, including type of anchors used, position of anchors, suture patterns and tensions, and other variables discussed herein. The system may also provide adaptive real time instructions based on unexpected issues that arise during surgery. For example, if the surgeon is having an issue with a preferred location of anchor placement, the system may recognize that issue in the training data, provide a best alternative placement position, where the best alternative placement position relies on data that lead to a positive outcome in similar situations.

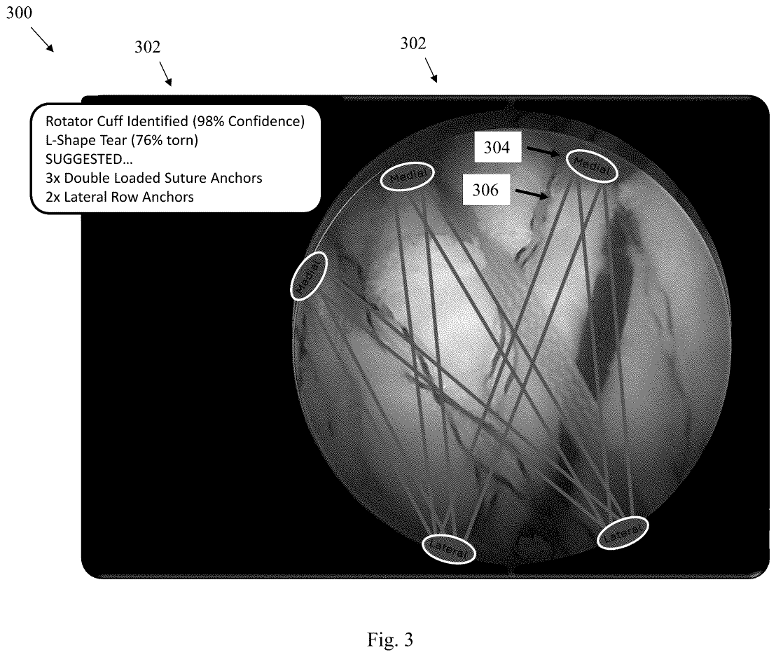

FIG. 3 is an image depicting an exemplary overlay or user interface (UI) 300 for an AI-OSS 200 that provides one or more outputs or interfaces to a user. In some embodiments, outputs 302 of the system comprise any of an AR video output, an overlay video output, augmented video output, AI or AR arthroscopic video overlay, one or more tissue or anatomical structure identification, identifiers, or graphical representations thereof, one or more treatment options, suture tension, change in suture tension, change in suture tension over time. In some embodiments, portions of the overlay or UI are translucent, transparent, masked or opaque, such as a translucent or unmasked central portion focusing on the region of interest in the subject. In some embodiments, the overlay or UI displays patient metrics, scorings, and treatment options or recommendations as discussed herein. In some embodiments, the UI 300 identifies suitable anchor locations 304 as well as suture arrangements 306 with one or more overlays. In some embodiments, the UI 300 identifies or marks high risk areas such as “do-not-drill” locations or areas, or proximity alerts or warning for vasculature and/or nerves based on the region of interest. In some embodiments, the AR, UI and/or overlays are provided to smart glasses and/or a HUD. In some embodiments, the smart glasses are configured to evaluate live imaging from any number of sensors (e.g., an arthroscope and/or camera(s) or sensors mounted on the smart glasses). In some embodiments, recommendations based on the live imaging may be provided to a surgeon with the smart glasses and/or HUD.

In some embodiments, a confidence score for an identified anatomical structure is calculated by the system and provided to the user. In some embodiments, the type of tear (e.g., L-Shape tear) is identified for one or more anatomical structure. In some embodiments, the percentage tear (e.g., 76% torn) is calculated by the system and provided to the user. In some embodiments, anchor arrangements, patterns (e.g., 2× lateral row anchor), loadings (e.g., double loaded suture anchors), or features are detected or identified by the system and provided to the user in the form of an overlay or list. In some embodiments, the system recommends treatment options such as anchor arrangements, patterns or loadings for anchors and/or suture to treat or repair the anatomical structure. In some embodiments, a pull-out risk score is calculated, and displayed, for each anchor based on the treatment option (e.g., selected anchor type and suture arrangement) and may be further based on a predicted or calculated bone density in the region of interest. In some embodiments, the system provides step-wise instructions to a physician to guide the treatment, such as recommending placement of anchors and/or suture based on a predetermined strategy.

In some embodiments, the inputs and/or outputs may also be used as training data for the disclosed models. In some embodiments, the training data may further comprise data scraped from of any previous publication or case history including surgical device requirement, surgical device type, closure device requirement, closure device type, tissue anchors required, tissue anchor type, tissue anchor location, tissue anchor layout, suture type, suture location, suture strength, and suture configuration; or inputs from a user including changes to the overlay, addition or removal of a closure device, addition or removal of an anchor, addition or removal of suture, change in closure device location, change in anchor location, change in suture location or configuration, addition or removal of an identified anatomical structure, refining the location or representation of an anatomical structure, addition or removal of an identified injury, refining the location or representation of an identified injury; or any of medical imaging data, raw arthroscopic video recordings, edited arthroscopic video recordings with manually added overlays, edited arthroscopic video recordings with identified anatomical structures or injuries, possible treatment options, treatment options employed in a repair, closure devices employed in a repair, anchors employed in a repair, anchor layout employed in a repair, suture used in a repair, suture configuration used in a repair, data scraped from online medical journal publications, case registries, patient information, and treatment outcomes.

Aspects of the invention relate to a machine learning algorithm, machine learning engine, machine learning model, or neural network. In various embodiments, the machine engine may include supervised, semi-supervised and unsupervised learning. In the case of supervised learning, algorithms may include nearest neighbor, naïve bayes, decision trees, support vector machines, and neural networks. In various embodiments, models may be trained by updating parameters based on various attributes of orthopedic surgery, for example surgical videos, patient information or imaging, or data scraped from online medical publications, and may output an augmented video, such as an arthroscopic video with overlays based on the attributes. In various embodiments, the supervised or semi-supervised model may be updated with hyperparameters for a deep learning model. In a preferred embodiment, a neural network may be trained by updating parameters based on various attributes of a patient, for example patient info, raw arthroscopic video of a portion of the patient, and the type of tear identified in the video, and may output an identified anatomical structure with a confidence score, a tear score or percentage for the injury, and/or one or more treatment options based on the attributes. In some embodiments, an ensemble model combines multiple models to formulate a more robust model that is less prone to errors. In some embodiments, a master model exists in a cloud instance, and a surgeon can choose to use the master model, or can choose and/or customize a local model.

In some embodiments, attributes may include patient demographics and medical history, including age, gender, height, weight, BMI, lifestyle factors (e.g., smoking status, activity level), relevant pre-existing conditions (e.g., diabetes, osteoporosis), previous surgical history or repairs; anatomical and structural data, including identified structures such as bones, tendons, ligaments, muscles, organs, blood vessels, nerve roots, abnormal tissues (e.g., tumors, tears, lesions, connective tissue disorders, soft-tissue injuries), injury characteristics such as tear type (e.g., L-shaped, bucket-handle), tear location, severity of injury (tear score, percentage, depth, extent), and changes in tissue appearance (color, shape, contour); surgical procedure attributes, including closure devices (e.g., tissue anchors, sutures, staples), anchor type, placement, orientation, layout, suture type, configuration (e.g., single-row, double-row), suture strength, tension, real-time adjustments to anchor or suture placement, and surgeon feedback or procedural changes during surgery; intraoperative measurements and real-time feedback, including suture tension measurements and changes over time, tissue reaction data (e.g., deformation under tension or stress), responses to procedural adjustments, movements detected in the region of interest, and confidence scores for detected structures or injuries; case outcomes and healing progression, including post-operative imaging (e.g., MRI, X-ray), patient recovery timelines, follow-up data, complication rates (e.g., re-tear, revision surgery), and healing rates correlated with anchor placement or suture configuration; overlay and visual representation attributes, including accuracy of AR overlay placements relative to anatomical structures, clarity and usability of graphical representations, adaptive changes in the overlay based on intraoperative findings, and overlay modifications such as refining the location or representation of anatomical structures and injuries; user interface interactions (e.g., surgeon commands or prompts), and time-stamped data capturing system adjustments during surgery; procedural and contextual data, including pre-operative imaging, patient-specific risk factors, surgical device cost data, intraoperative vital signs; medical training data, medical imaging data, raw and edited arthroscopic video recordings (with manually added overlays, identified structures, or injuries), possible treatment options, treatment options employed in repairs, closure devices, anchors, anchor layouts, suture configurations, data scraped from online medical journal publications, case registries, patient information, and treatment outcomes. Attributes given the heaviest weights may for example include medical imaging data, raw and edited arthroscopic video, and possible treatment options. The resulting identified anatomical structures, anatomical objects, medical devices, surgical devices, closure devices, injuries, or treatment options may then be judged according to one or more binary classifiers or quality metrics, and the weights of the attributes may be optimized to maximize the average binary classifiers or quality metrics. In this manner, a neural network can be trained to predict and optimize for any binary classifier or quality metric that can be experimentally measured. Examples of binary classifiers or quality metrics that a neural network can be trained on include success or failure of surgical outcome, presence or absence of complications (e.g., re-tear, infection), proper vs. improper anchor placement, proper vs. improper suture configuration, adequate vs. inadequate tissue healing rate, suture tension within acceptable range vs. outside range, correct vs. incorrect classification of anatomical structures, surgical devices, or injuries, accurate vs. inaccurate identification or recommendation of treatment options (e.g., anchor type, layout, suture configuration), high vs. low confidence score for identified anatomical structures or injuries, prediction of post-operative success vs. risk of complications (based on real-time and/or patient-specific data), accurate vs. inaccurate prediction or calculation of tear percentage and any other suitable type of quality metric that can be measured. In some embodiments, the neural network may have multi-task functionality and allow for simultaneous prediction and optimization of multiple quality metrics.

In embodiments that implement supervised or semi-supervised models, such as a neural network, a query may be performed in various ways. A query may request the neural network identify an anatomical structure, injury, or surgical device to increase a desirable parameter, for example the anatomical structure classification confidence score or one or more treatment options, or various treatment options with listed costs and predicted operative time. A supervised or semi-supervised learning model of the present invention may identify one or more anatomical structures, injuries or treatment options whose predicted confidence score or classification accuracy (as evaluated by the neural network) exceeds a predefined threshold, thereby indicating that the identified structure or injury is accurately classified and ready for overlay display or treatment planning. As contemplated herein, a predicted structure or injury may be any anatomical feature, injury type, or procedural element relevant to the orthopedic procedure.

In some embodiments, the supervised or semi-supervised learning model may be updated by training the model using a value of the desirable parameter associated with an input from a user or a surgeon, or associated with an anatomical structure, injury, or surgical device. In some embodiments, follow up data may be provided to the training data specific to a patient, patient outcome and/or treatment outcome for fine-tuning the model. In the case of the neural network, this may be referred to as the input layer. Updating the model in this manner may improve the ability of the model in proposing optimal treatment options and calculating scorings (e.g., anatomical structure confidence score, injury tear score). In the case of the neural network, this may be referred to as the output layer. In some embodiments, training the model may include using a value of the desirable parameter associated with a known injury or treatment outcome. For example, in some embodiments, training the model may include predicting a value of the desirable parameter for the proposed treatment option, comparing the predicted value to the corresponding value associated with a known treatment outcome, and training the model based on a result of the comparison. If the predicted value is the same or substantially similar to the observed value, then the neural network may be minimally updated or not updated at all. If the predicted value differs from that of the known treatment outcomes, then the model may be substantially updated to better correct for this discrepancy. Regardless of how the model is retrained, the retrained model may be used to propose additional treatment options, or be used to identify injuries, identify anatomical structures, and identify surgical devices.

Although the techniques of the present application are in the context of AI assisted orthopedic surgery, it should be appreciated that this is a non-limiting application of these techniques as they can be applied to other types of parameters or attributes, for example related to cardiac, neuro, gastrointestinal, vascular, pulmonology, rheumatology, dermatology, urology, gynecology, obstetrics, ophthalmology and dentistry fields. Depending on the type of data used to train the model, the neural network can be optimized for different types of surgical applications. In this manner, a neural network can be trained to provide treatment options beyond orthopedic procedures and tissue injuries.

The techniques described herein associated with iteratively querying a model by inputting an image of a region of interest in a subject, receiving an output from the model that has an identified anatomical structure with a confidence score, a tear score or percentage, or one or more treatment options based on the attributes, and successively providing an identified anatomical structure, a tear score, or one or more treatment options as an input to the model, can be applied to other machine learning applications. Such techniques may be particularly useful in applications where a final output having a desirable patient outcome, low complication rate, short operative time, and/or low cost of medical device(s) is desired. Such techniques can be generalized for identifying a series of discrete attributes by applying a model generated by a model trained using data relating the discrete attributes to a characteristic of a series of the discrete attributes. In the context of desirable treatment outcomes, the discrete attributes may include complication rates (e.g., re-tear, revision surgery), healing rates, or any disclosed training metric or quality metric.

In some embodiments, an iterative process or parameter updating is formed by querying the model for a desirable treatment outcome, receiving one or more treatment options, and identifying a preferred treatment option based on the desired treatment outcome. An additional iteration of the iterative process may include inputting the treatment option or outcome from an immediately prior iteration. The iterative process may stop when a treatment outcome matches a prior desirable treatment outcome from the immediately prior iteration. In the case of the neural network, the parameters are updated for the hidden layer, where the hidden layer is positioned between the input and output layers discussed above. The hidden layer of the neural network may include any number of layers or depth. Each of the layers may be associated with a number of nodes or width.

The disclosed system and method may also be used as an internal research tool for design efforts to bring about new medical device technology. For example, simulated treatments, existing devices, and prototype devices may be used with the system and method to test and iterate on designs. In some embodiments, device type (e.g., anchor type), suture type, suture layout, and suture configuration as applied to a specific injury or region of interest on a subject may be tested or simulated to predict treatment and patient outcomes, and optimize treatment methods and device design.

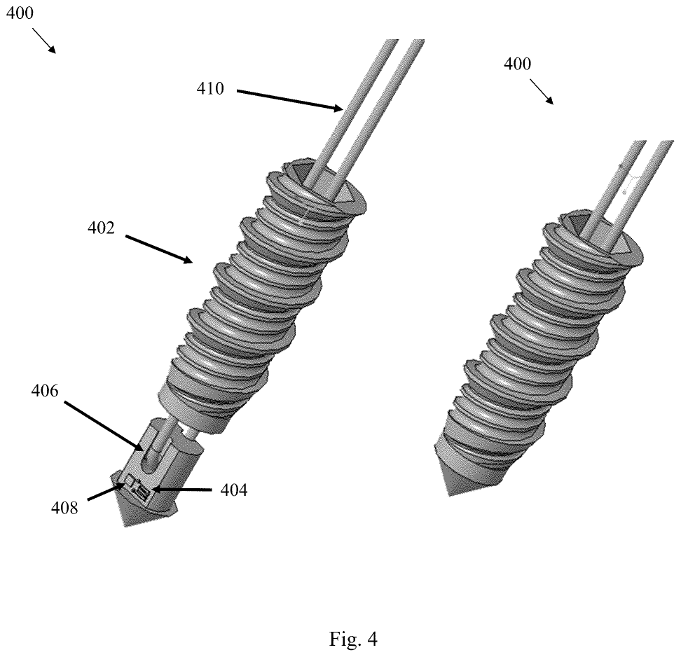

An exemplary active implant or sensing tissue anchor 400 (e.g., smart anchor) is disclosed herein. In some embodiments, the anchor 400 comprises an integrated strain gauge 404 that can relay suture 410 tension outside the body 402. FIG. 4 depicts an exemplary pressure sensing anchor 400 that may be used in conjunction with any disclosed device, system and/or method. In some embodiments, the strain gauge 404 is applied or formed in the suture eyelet 406 stem of the anchor. In some embodiments, the anchor 400 is configured to wirelessly communicate suture tension (e.g., with a wireless transmitter 408) to the disclosed system, however it may function with any suitable software and interface for relaying measurements captured by the device to a user. In some embodiments, the anchor or disclosed system measures suture tension, or change in suture tension. In some embodiments, change in tension could indicate any of suture tearing through tendon, suture cutting through bone, and/or tendon healing. Advantages of such an anchor include real time feedback on tendon repair. In some embodiments, the anchor measures any of compressive load of tendon on top of implant, temperature, movement, orientation, vibration, bony ingrown into the anchor, or the like. In some embodiments, one or more anchor 400 may be used to calculate load imbalance on the anchors/and or suture, and the system 200 may provide recommendations to add or change treatment options (e.g., suture tension) based on the calculated loads.

Any exemplary “smart device” (e.g., smart anchor) is disclosed, however it should be appreciated that the disclosed system and methods thereof may be used with any smart medical devices, smart feedback medical devices, smart implants, and other sensory/feedback medical devices that would be known by one of normal skill in the art. This includes, but is not limited to, smart anchors, smart grafts, smart scaffolds, smart buttons, smart suture, smart implants, smart knee implants, smart hip implants, smart joint implants, smart spinal implants, smart fracture fixation devices, and the like. These “smart” devices may communicatively connect to the disclosed system and provide sensory data and feedback similar as to described for anchor 400 above, such as physical parameters like pressure, force, strain, displacement, proximity and temperature.

The disclosed system and method may be used with devices, methods and treatments for soft-tissue repair. However, the disclosed system and method may also be used with any orthopedic devices, implants, prosthesis, and the like, and any methods of use or treatment associated therewith. For example, the system and method may be used for repair of bone or bony structures, or in open repairs. In an example embodiment, the disclosed system may collect imaging data of a region of interest of a subject comprising one or more bones, analyze the imaging data with a neural network, wherein the neural network is trained to identify anatomical structures such as bones and joints, surgical devices and injuries, classify, by the neural network, at least one of anatomical structures, surgical devices, and injuries in the imaging data, generate an overlay for the video with one or more graphical representations of any classified at least one anatomical structures, surgical devices and injuries, and output one or more treatment options for any identified injury, wherein the treatment options comprise any of screws, plates, rods, nails, wires, pins, bolts, bone growth stimulation, cutting, bending, burring, implant, immobilization, setting, and casting, and the like.

Any of the following systems, devices, anchors, scaffolds, sutures and the like, and any methods thereof, may be used with the disclosed system and method. The following publications are hereby incorporated by reference in their entirety:
U.S. Non-Provisional application Ser. No. 19/286,727, filed on Jul. 31, 2025 “Knotless Anchor System and Method”.
U.S. Provisional Application No. 63/748,290, filed on Jan. 22, 2025 “Bone Screw and Method of Repairing Soft Tissue”.
U.S. Patent Publication No. US 2025/0090160 A1 “Suture anchor device, system and method of use”.

U.S. Patent Publication No. US 2024/0165300 A1 “3d printed scaffolds for use in tissue repair”.

U.S. Patent Publication No. US 2024/0090889 A1 “Implant device, system and method”.

U.S. Patent Publication No. US 2024/0225631 A1 “Labral Anchor System and Method”.

U.S. Patent Publication No. US 2024/0130722 A1 “Methods for attaching tissue to bone utilizing a scaffold”.

U.S. Patent Publication No. US 2023/0157681 A1 “Minimally invasive anchor drill systems”.

U.S. Patent Publication No. US 2022/0287707 A1 “Scaffold and Suture Anchoring Device”.

U.S. Patent Publication No. U.S. Pat. No. 12,310,574 B2 “Knotless suture anchor with re-tension features”.

U.S. Patent Publication No. US 2021/0153861 A1 “Fastener anchoring device”.

U.S. Patent Publication No. US 2018/0360438 A1 “Suture anchor with microthreads and suture anchor driver with needle attachment”.

U.S. Patent Publication No. US 2015/0127048 A1 “Apparatus and Method for Securing Tissue to Bone Using Suture Anchors with a Pre-Loaded Piercing Structure and Sutures”.
Ledet, Eric H., et al. “Smart implants in orthopedic surgery, improving patient outcomes: a review.” Innovation and entrepreneurship in health (2018): 41-51.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method for assisting orthopedic surgery, comprising the steps of:
collecting imaging data of a region of interest of a subject;
analyzing the imaging data with a neural network, wherein the neural network is trained to identify anatomical structures, surgical devices and injuries;
classifying, by the neural network, at least one of anatomical structures, surgical devices, and injuries in the imaging data;
generating an overlay for the video with one or more graphical representations of any classified at least one anatomical structures, surgical devices and injuries; and
outputting one or more treatment options for any identified injury.

2. The method of claim 1, wherein the one or more treatment options are selected from the group consisting of non-implant repair, surgical device requirement, surgical device type, closure device requirement, closure device type, tissue anchor requirement, tissue anchor type, tissue anchor number, one or more tissue anchor locations, tissue anchor layout, suture type, suture locations, suture configuration, scaffold requirement, scaffold type, scaffold number, scaffold material, scaffold location, scaffold configuration, button requirement, button type, button number, button material, button location, button configuration, plate requirement,

3. The method of claim 1, wherein the overlay displays one or more treatment options comprising graphical representations of a non-implant repair, one or more surgical devices, closure devices, tissue anchors or suture applied to treat an identified injury.

4. The method of claim 3, wherein the overlay displays one or more graphical representations of suture attached to one or more tissue anchors applied to treat an identified injury.

5. The method of claim 4, wherein each graphically represented anchor or suture is numbered, or labelled with a position relative to the region of interest, injury, or anatomical structure.

6. The method of claim 1, further comprising the steps of:
receiving one or more inputs from a user; and
modifying at least one of the overlay or the one or more treatment options based on the inputs from the user.

7. The method of claim 6, wherein the one or more inputs are selected from the group consisting of changes to the overlay, addition or removal of a closure device, addition or removal of an anchor, addition or removal of suture, change in closure device location, change in anchor location, change in suture location or configuration, addition or removal of an identified anatomical structure, refining the location or representation of an identified anatomical structure, addition or removal of an identified injury, refining the location or representation of an identified injury, preoperative MRI data, patient examination data, quality of tendon, excursion, characteristics of tear, degree of retraction, and superior migration.

8. The method of claim 1, wherein the neural network is trained with medical training data selected from the group consisting of medical imaging data, raw arthroscopic video recordings, edited arthroscopic video recordings with manually added overlays, edited arthroscopic video recordings with identified anatomical structures or injuries, MRI data, preoperative MRI data, postoperative MRI data, possible treatment options, treatment options employed in a repair, closure devices employed in a repair, anchors employed in a repair, anchor layout employed in a repair, suture used in a repair, suture configuration used in a repair, data scraped from online medical journal publications, case registries, patient information, patient outcomes, and treatment outcomes.

9. The method of claim 1, further comprising the steps of:
collecting patient information of the subject;
analyzing the patient information with the neural network; and
modifying at least one of the overlay or the one or more treatment options based on the results from the neural network.

10. The method of claim 9, further comprising the steps of:
predicting post-operative outcomes based on the one or more treatment options and the patient information; and
displaying the predicted outcomes in the overlay to provide real-time guidance to the user.

11. The method of claim 1, further comprising the steps of:
calculating an anatomical structure identification confidence score; and
displaying the confidence score in the overlay.

12. The method of claim 1, further comprising the steps of:
calculating a tear percentage of an identified injury; and
displaying the tear percentage in the overlay.

13. The method of claim 1, further comprising the steps of:
monitoring real-time changes in the region of interest with the neural network; and
modifying at least one of the overlay or the one or more treatment options based on the changes.

14. The method of claim 13, wherein the real-time changes comprise any of tissue deformation, tissue color change, movement of anatomical structures, movement of surgical devices, movement of closure devices, anchor position, suture tension, and suture position.

15. The method of claim 1, further comprising the step of receiving signals from one or more pressure sensing tissue anchors configured to measure tension of suture attached to the anchor.

16. The method of claim 15, further comprising the step of displaying the measured tension in the overlay.

17. The method of claim 15, further comprising the step of modifying the one or more treatment options based on the measured tension.

18. The method of claim 15, further comprising the step of measuring change in suture tension over time.

19. The method of claim 18, wherein a change in suture tension over time may be representative of suture tearing through tendon, suture cutting through bone, and tendon healing.

20. The method of claim 1 further comprising:
outputting one or more alternative treatment options based on an input indicative of an issue with the one or more treatment options.

21. The method of claim 1, wherein the one or more treatment options includes a non-implant repair comprising one or more procedures selected from consisting of clean up of tendon, clean up of surfaces, acromioplasty to address impingent.

22. The method of claim 1, wherein the imaging data comprises at least one of camera image, sensor image, camera video, sensor video, live camera video, live sensor video, endoscopic image, endoscopic video, live endoscopic video, prerecorded endoscopic video, arthroscopic image, arthroscopic video, live arthroscopic video, prerecorded arthroscopic video, non-arthroscopic video, wearable device camera image, wearable device sensor image, wearable device camera video, wearable device sensor video, smart glasses camera image, smart glasses sensor image, smart glasses camera video, smart glasses sensor video, live smart glasses camera video, live smart glasses sensor video, surgical camera, operating room camera.

23. An AI assisted orthopedic surgery system comprising a non-transitory computer-readable medium with instructions stored thereon, that when executed by a processor perform the steps of:
collecting imaging data of a region of interest of a subject;
analyzing the imaging data with a neural network, wherein the neural network is trained to identify anatomical structures, surgical devices and injuries;
classifying, by the neural network, at least one of anatomical structures, surgical devices, and injuries in the imaging data;
generating an overlay for the video with one or more graphical representations of any classified at least one anatomical structures, surgical devices and injuries; and
outputting one or more treatment options for any identified injury.

24. The system of claim 23, wherein the system is configured to connect between an arthroscopic camera and a display.

25. The system of claim 23, further comprising one or more pressure sensing tissue anchors connected to the system configured to measure tension of suture attached to the anchor.
